(19) United States
(12) Patent Application Publication
(10) Pub. No.: US 2011/0276058 A1
(43) Pub. Date: Nov. 10, 2011

(54) SURGICAL ROBOT SYSTEM, AND METHOD FOR CONTROLLING SAME

(76) Inventors: Seung Wook Choi, Gyeonggi-do (KR); Jong Seok Won, Gyeonggi-do (KR); Min kyu Lee, Gyeonggi-do (KR); Bae Sang Jang, Gyeonggi-do (KR); Woo Jyoung Lee, Seoul (KR)

(21) Appl. No.: 13/144,427

(22) PCT Filed: Feb. 8, 2010
(86) PCT No.: PCT/KR2010/000761

§ 371 Date: Jul. 13, 2011

(30) Foreign Application Priority Data
Feb. 11, 2009 (KR) 10-2009-0011140

(51) Int. Cl. A61B 19/00

(52) U.S. Cl. 606/130

(57) ABSTRACT

A master interface for a surgical robot, mounted on a master robot for controlling a slave robot, may include two or more robot arms each having a mounted surgical instrument. The master interface may include: a screen display unit configured to display an on-screen image corresponding to a picture signal inputted from a surgical endoscope; two or more arm manipulation units equipped for controlling the two or more robot arms, respectively; and a control unit configured to provide control such that the on-screen image is rotated or mirrored in a pre-designated direction according to a user manipulation, and configured to provide control such that control conditions for the robot arm are renewed to match the rotated or mirrored on-screen image. Thus, the display screen on a surgical monitor can be suitably controlled according to the intent of the surgeon, to remove the non-intuitiveness of a surgical procedure.

### CROSS-REFERENCE TO RELATED APPLICATIONS

This application is the National Phase of PCT/KR2010/000761 filed on Feb. 8, 2010, which claims priority under 35 U.S.C. 119(a) to Patent Application No. 10-2009-0011140 filed in the Republic of Korea on Feb. 11, 2009, all of which are hereby expressly incorporated by reference into the present application.

### BACKGROUND

The present invention relates to surgery, more particularly to a surgical robot and a control method thereof.

A surgical robot refers to a robot that has the capability to perform a surgical action in the stead of a surgeon. The surgical robot may provide the advantages of accurate and precise movements compared to a human and of enabling remote surgery.

Some of the surgical robots currently under development around the globe include bone surgery robots, laparoscopic surgery robots, stereotactic surgery robots, etc. Here, a laparoscopic surgical robot is a robot that performs minimally invasive surgery using a laparoscope and a miniature surgical instrument.

Laparoscopic surgery is a cutting-edge technique that involves perforating a hole of about 1 cm in the navel area and inserting a laparoscope, which is an endoscope for looking inside the abdomen. Further advances in this technique are expected in the future.

Current laparoscopes are mounted with computer chips and have been developed to the extent that magnified pictures can be obtained that are clearer than pictures seen with the naked eye, and when used with specially-designed laparoscopic surgical instruments while looking at a monitor screen, any type of surgery is possible.

Moreover, despite the fact that its surgical range is almost equal to that of laparotomy surgery, laparoscopic surgery produces fewer complications than does laparotomy, enables treatment within a much shorter time after the procedure, and helps the surgery patient maintain his/her stamina or immune functions. As such, laparoscopic surgery is being established as the standard surgery for treating colorectal cancer, etc., in places such as America and Europe.

However, laparoscopic surgery may entail certain difficulties, because a laparoscopic operation entails laparoscopic surgical instruments that are not as familiar to use as their counterparts in laparotomy surgery, 2-dimensional pictures, and mirror images, and because the surgery cannot be performed while touching with one's own hands.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the invention is to provide a surgical robot system and a method of controlling the surgical robot system, in which the display screen on a surgical monitor can be suitably controlled according to the intent of the surgeon, to remove the non-intuitiveness of a surgical procedure.

Also, an aspect of the invention is to provide a surgical robot system and a method of controlling the surgical robot system, in which the robot arm can be controlled in a way matching the control of the display screen through the surgical monitor, so that an operator may perform a surgical procedure smoothly and intuitively.

Another aspect of the invention provides a master interface for a surgical robot, where the master interface is mounted on a master robot for controlling a slave robot, which includes two or more robot arms each having a mounted surgical instrument. The master interface includes: a screen display unit configured to display an on-screen image corresponding to a picture signal inputted from a surgical endoscope; two or more arm manipulation units equipped for controlling the two or more robot arms, respectively; and a control unit configured to provide control such that the on-screen image is rotated or mirrored in a pre-designated direction according to a user manipulation, and configured to provide control such that control conditions for the robot arm are renewed to match the rotated or mirrored on-screen image.

The surgical endoscope can be one or more of a laparoscope, a thoracoscope, an arthroscope, and a rhinoscope.

The master interface can further include a manipulation signal generator unit, which may generate a manipulation signal according to a user manipulation on the arm manipulation unit and transmit the manipulation signal to the slave robot, and which may generate a manipulation signal for one or more of a position adjustment for one or more of a robot arm and a surgical instrument and a changing of the arm manipulation units corresponding respectively to the robot arms in order to renew the control conditions.

The manipulation signal generator unit can perform an arm manipulation change according to display-based instrument control. Here, the display-based instrument control can include generating a manipulation signal for renewing a configuration such that a surgical instrument located on a right side in the rotated on-screen image is manipulated by an arm manipulation unit located on a right side of an operator, and a surgical instrument located on a left side in the rotated on-screen image is manipulated by an arm manipulation unit located on a left side of an operator.

The manipulation signal generator unit can generate a manipulation signal for adjusting a position of one or more of a robot arm and a surgical instrument in a certain direction in accordance with a user manipulation for the position adjustment regardless of a rotation of the on-screen image.

The manipulation signal generator unit can generate a manipulation signal for having the surgical instrument move in a rotational movement in the on-screen image to match a rotation angle of the on-screen image.

The user manipulation can be a manipulation by a user voice, and the control unit can receive the user voice as input, perform recognition and analysis, and then control the on-screen image to rotate in a pre-designated direction.

The control unit can compute a rotation angle by instrument-based display control and can provide control such that the on-screen image is displayed rotated to match the computed rotation angle. The instrument-based display control can be for forming a virtual trapezoid or quadrilateral having two surgical instruments as oblique sides and computing the rotation angle such that a top side or a bottom side of the trapezoid or quadrilateral becomes parallel to a horizontal plane of a display screen within a margin of error.

The master interface can further include a screen-rotation manipulation unit that is configured to receive as input the user manipulation for rotating the on-screen image in a pre-designated direction.

The on-screen image can be displayed rotated by a pre-designated rotation angle in proportion with a number of manipulations on the screen-rotation manipulation unit. The on-screen image can be displayed rotated along a pre-designated rotation direction in a certain rotating speed during a continuous manipulation holding time on the screen-rotation manipulation unit. The screen-rotation manipulation unit can be any one of a pedal, a clutch button, and a voice recognition device.

If there are position sensors located on multiple locations of the robot arm, the control unit can recognize an extending direction of the robot arm by using a sensing value obtained by each of the position sensors and can thereby compute a rotation angle of the on-screen image. In this case, the control unit can compute the rotation angle such that a front of the robot arm along the extending direction is positioned facing a particular direction (e.g. an upward direction) on a display screen.

The control unit can also compute a rotation angle of the on-screen image by using an extending direction of the robot arm recognized by analyzing an image taken by a camera equipped on a ceiling. In this case, a paint having one or more of a pre-designated color and a pre-designated material can be coated over any one of an entire area of, an upper area of, and a plurality of locations of the robot arm.

In order that the on-screen image may be displayed rotated to match the rotation angle, the surgical endoscope can be controlled to rotate about an axis formed along its extending direction.

The control unit can provide control such that a direction indicator is further displayed through the screen display unit, to provide information related to a rotation of the on-screen image. The direction indicator can be composed of one or more of: at least one character indicating a direction, a three-dimensional shape being rotated to match a rotation of the on-screen image, a development drawing of a three-dimensional shape including at least one block, and a compass, etc.

When the on-screen image is displayed in a mirrored configuration, the control unit can provide control such that one or more of a direction indicator, a warning message, and cautionary information is further displayed through the screen display unit. The mirroring of the on-screen image can be either left-right mirroring or up-down mirroring, and the cautionary information can include a boundary image of a pre-designated color for the on-screen image. The slave robot and the master robot can be integrated into a single body.

Still another aspect of the invention provides a method of controlling a surgical robot system that is performed in a master robot for controlling a slave robot, which includes two or more robot arms each having a mounted surgical instrument. This method includes: displaying an on-screen image corresponding to a picture signal inputted from a surgical endoscope; receiving as input a manipulation command for instructing a rotation or a mirroring of the on-screen image; and providing control such that the on-screen image is rotated or mirrored in a pre-designated direction according to the manipulation command and such that a control condition of the robot arm is renewed to match the rotated or mirrored on-screen image.

The providing of control such that a control condition of the robot arm is renewed can include: generating a manipulation signal for one or more of a position adjustment for one or more a robot arm and a surgical instrument and a changing of arm manipulation units corresponding respectively to the robot arms; and transmitting the manipulation signal to the slave robot.

The changing of the arm manipulation units can be performed according to display-based instrument control.

The display-based instrument control can include renewing a configuration such that a surgical instrument located on a right side in the rotated on-screen image is manipulated by an arm manipulation unit located on a right side of an operator, and a surgical instrument located on a left side in the rotated on-screen image is manipulated by an arm manipulation unit located on a left side of an operator.

A manipulation signal can be generated for adjusting a position of one or more of a robot arm and a surgical instrument in a certain direction in accordance with a user manipulation for the position adjustment regardless of a rotation of the on-screen image.

The position adjustment can include moving the surgical instrument in a rotational movement in the on-screen image to match a rotation angle of the on-screen image.

The rotation angle of the on-screen image can be determined in proportion to a number of times the manipulation command is inputted. Alternatively, the rotation angle of the on-screen image can be determined in proportion to a holding time during which the manipulation command is inputted.

The receiving of the manipulation command as input can include: receiving as input a user voice; and generating the manipulation command by recognizing and analyzing the inputted user voice. The manipulation command can be inputted by way of any one of a pedal, a clutch button, and a voice recognition device. The surgical endoscope can be one or more of a laparoscope, a thoracoscope, an arthroscope, and a rhinoscope.

The receiving of the manipulation command as input can include: receiving as input a sensing value from each of position sensors located on a plurality of locations of the two or more robot arms; computing a rotation angle for rotating the on-screen image by using the inputted sensing values to recognize an extending direction of the robot arms; and receiving the computed rotation angle as the manipulation command. Here, the rotation angle can be computed such that a front of one or more of the robot arm along the extending direction is positioned facing a particular direction (e.g. an upward direction) on a display screen.

The receiving of the manipulation command as input can include: receiving as input an image taken by a camera equipped on a ceiling; computing a rotation angle for rotating the on-screen image by using an extending direction of the robot arms as recognized from analyzing the inputted image; and receiving the computed rotation angle as the manipulation command. Here, a paint having one or more of a pre-designated color and a pre-designated material can be coated over any one of an entire area of, an upper area of, and a plurality of locations of the robot arm.

The surgical endoscope can be rotated about an axis formed along its extending direction so that the on-screen image is displayed rotated to match the rotation angle.

In providing the control, a direction indicator can be displayed to provide information related to a rotation of the on-screen image.

The direction indicator can be composed of one or more of: at least one character indicating a direction, a three-dimensional shape being rotated to match a rotation of the on-screen image, a development drawing of a three-dimensional shape including at least one block, and a compass.

In providing control such that the on-screen image is mirrored in a pre-designated direction, one or more of a direction indicator, a warning message, and cautionary information may further be displayed.

The mirroring of the on-screen image can be either left-right mirroring or up-down mirroring, and the cautionary information can include a boundary image of a pre-designated color for the on-screen image.

Yet another aspect of the invention provides a method of controlling a surgical robot system that is performed in a master robot for controlling a slave robot, which includes two or more robot arms each having a mounted surgical instrument. This method includes: displaying an on-screen image corresponding to a picture signal inputted from a surgical endoscope; computing a rotation angle for the on-screen image by instrument-based display control; and providing control such that the on-screen is displayed rotated by the computed rotation angle.

The instrument-based display control can be for forming a virtual trapezoid or quadrilateral having two surgical instruments as oblique sides and computing the rotation angle such that a top side or a bottom side of the trapezoid or quadrilateral becomes parallel to a horizontal plane of a display screen within a margin of error.

The surgical endoscope can be one or more of a laparoscope, a thoracoscope, an arthroscope, and a rhinoscope. In order that the on-screen image may be displayed rotated to match the rotation angle, the surgical endoscope can be controlled to rotate about an axis formed along its extending direction.

Additional aspects, features, and advantages, other than those described above, will be apparent from the drawings, claims, and written description below.

### BRIEF DESCRIPTION OF THE DRAWINGS

FIG. 1 is a plan view illustrating the overall structure of a surgical robot according to an embodiment of the invention.

FIG. 2 is a conceptual drawing illustrating a master interface for a surgical robot according to an embodiment of the invention.

FIG. 3 is a block diagram schematically illustrating the configuration of a master robot and a slave robot according to an embodiment of the invention.

FIG. 4 illustrates an example of adjusting the display of an on-screen according to an embodiment of the invention.

FIG. 5 illustrates an example of manipulating the arm manipulation units while viewing an original on-screen image that has not been rotated, according to an embodiment of the invention.

FIG. 6 and FIG. 7 illustrate examples of manipulating the arm manipulation units viewing an on-screen image that has been rotated 180 degrees, according to the related art.

FIG. 8 illustrates an example of manipulating the arm manipulation units while viewing an on-screen image that has been rotated 180 degrees, according to an embodiment of the invention.

FIG. 9 through FIG. 12 illustrate examples of displaying a direction indicator in correspondence to a rotation of the on-screen image, according to various embodiments of the invention.

FIG. 13 and FIG. 14 illustrate examples of displaying cautionary information in correspondence to a mirroring of the on-screen image, according to various embodiments of the invention.

FIG. 15 is a flowchart illustrating a method of controlling a display screen according to an embodiment of the invention.

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention. In the written description, certain detailed explanations of related art are omitted when it is deemed that they may unnecessarily obscure the essence of the present invention.

While such terms as “first” and “second,” etc., may be used to describe various components, such components must not be limited to the above terms. The above terms are used only to distinguish one component from another.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

Certain embodiments of the present invention will be described below in detail with reference to the accompanying drawings. Those components that are the same or are in correspondence are rendered the same reference numeral regardless of the figure number, and redundant descriptions are omitted.

Although the spirit of the invention can be generally applied to surgical operations in which a surgical endoscope (e.g. a laparoscope, thoracoscope, arthroscope, rhinoscope, etc.) is used, the embodiments of the invention will be described, for convenience, using examples in which a laparoscope is used.

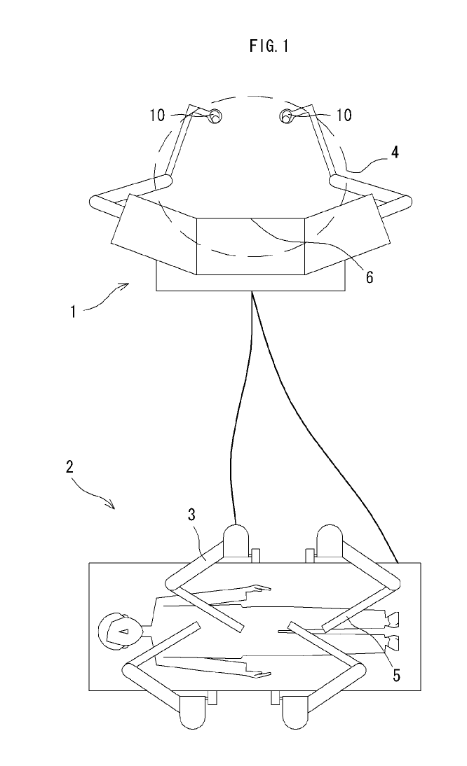

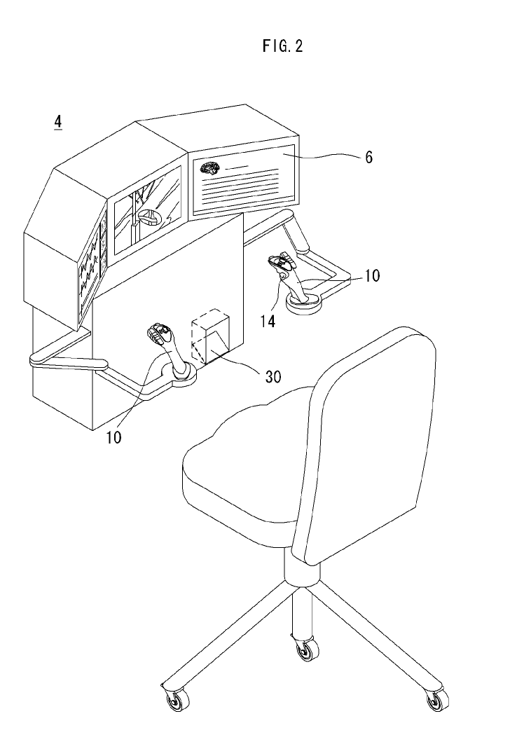

FIG. 1 is a plan view illustrating the overall structure of a surgical robot according to an embodiment of the invention, and FIG. 2 is a conceptual drawing illustrating a master interface for a surgical robot according to an embodiment of the invention.

Referring to FIG. 1 and FIG. 2, a robot system for laparoscopic surgery may be include a slave robot 2, which performs surgery on a patient lying on the operating table, and a master robot 1, by which the operator remotely controls the slave robot 2. The master robot 1 and slave robot 2 do not necessarily have to be physically separated as independent individual devices, but can be integrated into a single body, in which case a master interface 4 can correspond to the interface portion of the integrated robot.

The master interface 4 of the master robot 1 may include a monitor 6, handles 10, and display screen control buttons. A display screen control button can be implemented in the form of a clutch button 14 or a pedal 30, and if the clutch button 14 and the pedal 30 are implemented to perform the same function, then it is also possible to have just one of the clutch button 14 and pedal 30 included in the master interface 4. The slave robot 2 may include robot arms 3 and laparoscopes 5.

The master interface 4 may include two handles 10, so that the operator may perform the manipulations with the handles held in both hands, and the manipulation signals resulting from the manipulation by the operator on the handles 10 may be transmitted to the slave robot 2 to control the robot arms 3.

On the monitor 6 of the master interface 4, an image inputted by the laparoscope 5 may be displayed as an on-screen image. The monitor 6 can also additionally display the patient's cardiogram, etc., as shown in the example.

The on-screen image displayed on the monitor 6 can be controlled, by a manipulation by the operator on the display screen control button, to be rotated in a clockwise or counterclockwise direction by a designated rotation angle. This is to remove the inconvenience of having to perform surgery while viewing an on-screen image that is not intuitive because the direction in which the operator performs surgery with respect to a surgical site does not coincide with the direction of the on-screen image, in cases where an image is inputted to a laparoscope 5 having a fixed position and transmitted to the master interface 4. Of course, even in cases where the position of the laparoscope 5 can be adjusted by a manipulation on a clutch button, etc., equipped on a handle 10, it is also possible to control the display direction to be rotated in a clockwise or counterclockwise direction by a manipulation on the pedal 30, if the on-screen image displayed on the monitor 6 is of a particular arrangement.

Also, if the display arrangement of the on-screen image displayed on the monitor 6 is changed by a manipulation by the operation on the display screen control button, the positions or functions of the robot arms 3 can be adjusted correspondingly to allow a more intuitive surgery procedure for the operator.

As described above, a feature of this embodiment is to enable the operator to alter the on-screen image to a desired display arrangement (for example, to rotate by a particular angle, etc.) by manipulating a display screen control button, regardless of the position of the slave robot 2. Another feature is to adjust the positions or functions of the robot arms 3 on the slave robot 2 such as to allow intuitive recognition by the operator.

The slave robot 2 and the master robot 1 can be interconnected by a wired or a wireless network to exchange manipulation signals, etc., with each other. If there are two manipulation signals originating from the two handles 10 equipped on the master interface 4 and/or a manipulation signal for a position adjustment of the laparoscope 5 that have to be transmitted simultaneously and/or at a similar time, each of the manipulation signals can be transmitted to the slave robot 2 independently of one another. Here, to state that each manipulation signal may be transmitted “independently” means that there is no interference between manipulation signals and that no one manipulation signal affects another signal. Various methods can be used to transmit the multiple manipulation signals independently of one another, such as by transmitting the manipulation signals after adding header information for each manipulation signal during the generating the manipulation signals, transmitting the manipulation signals in the order in which they were generated, or pre-setting a priority order for transmitting the manipulation signals, and the like. It is also possible to fundamentally prevent interference between manipulation signals by having independent transmission paths through which the manipulation signals may be transmitted respectively.

The robot arms 3 of the slave robot 2 can be implemented to have high degrees of freedom. A robot arm 3 can include, for example, a surgical tool that will be inserted in the surgical site of the patient, a yaw driving unit for rotating the surgical tool in a yaw direction according to the operating position, a pitch driving unit for rotating the surgical tool in a pitch direction perpendicular to the rotational driving of the yaw driving unit, a transport driving unit for moving the surgical tool along a lengthwise direction, a rotation driving unit for rotating the surgical tool, and a surgical tool driving unit installed on the end of the surgical tool to incise or cut a surgical lesion. However, the composition of the robot arms 3 is not thus limited, and it is to be appreciated that such an example does not limit the scope of claims of the present invention. The actual control procedures by which the robot arms 3 are rotated, moved, etc., in correspondence to the operator manipulating the handles 10 will not be described here in detail, as they are not directly connected with the essence of the invention.

One or more slave robots 2 can be used to perform surgery on a patient, and the laparoscope 5 for displaying the surgical site on the monitor 6 as an on-screen image can be implemented on an independent slave robot 2.

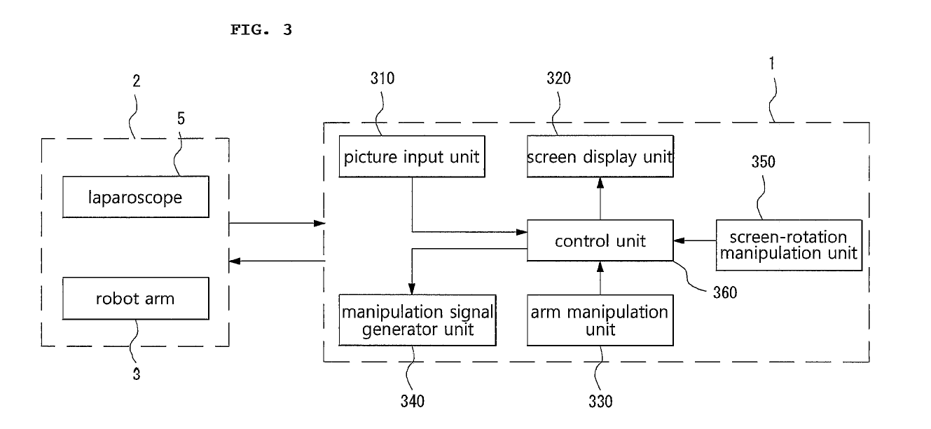

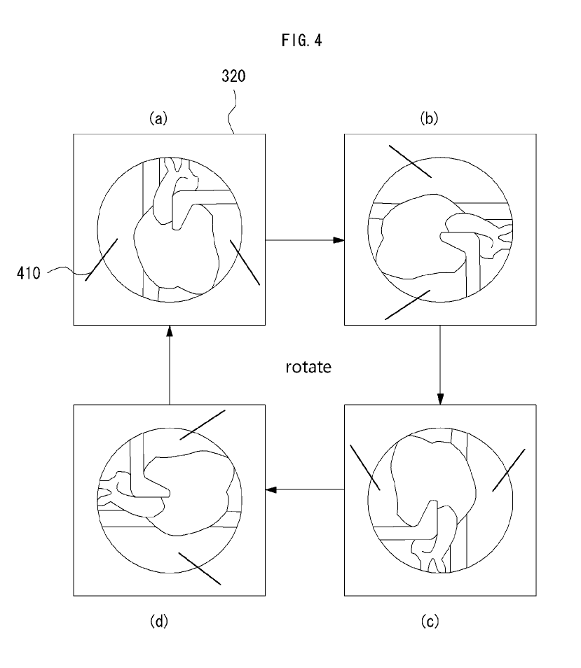

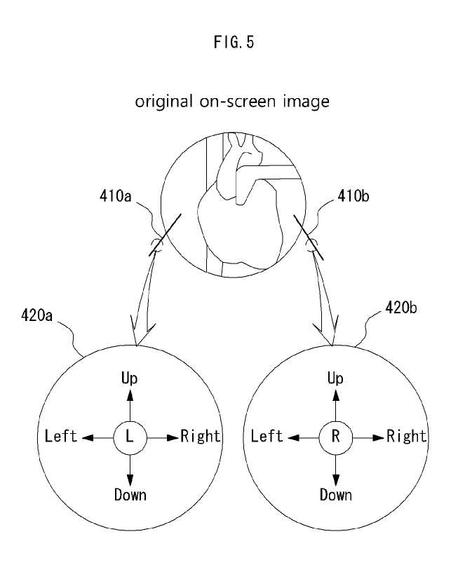

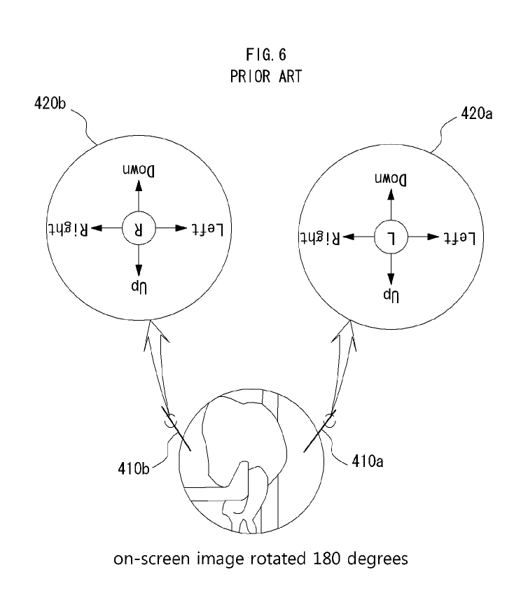

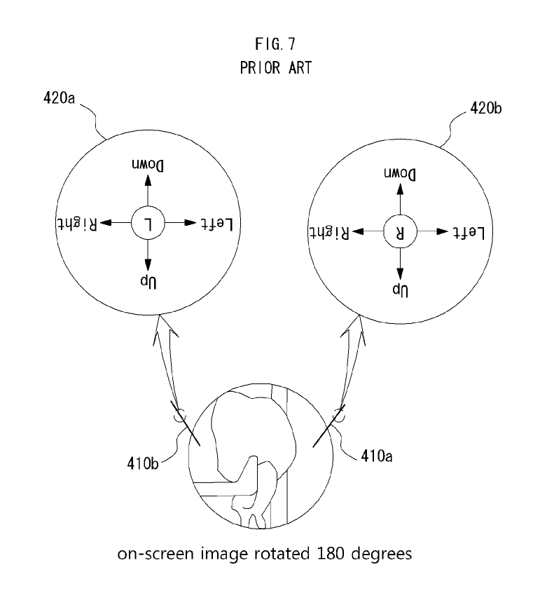

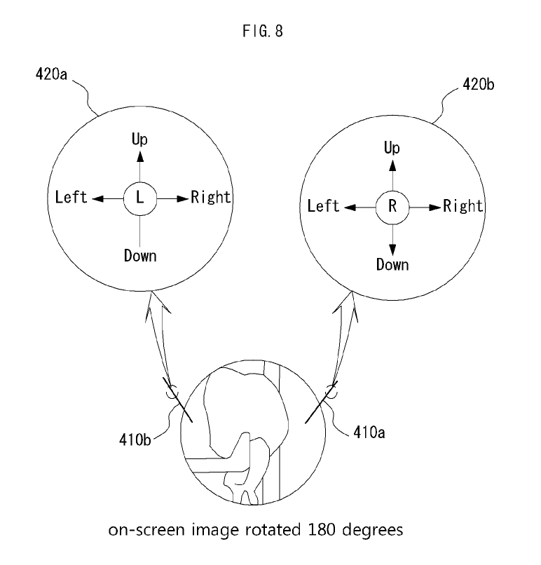

FIG. 3 is a block diagram schematically illustrating the configuration of a master robot and a slave robot according to an embodiment of the invention, and FIG. 4 is an illustration of an example of adjusting the display of an on-screen according to an embodiment of the invention. FIG. 5 illustrates an example of manipulating the arm manipulation units while viewing an original on-screen image that has not been rotated according to an embodiment of the invention, FIG. 6 and FIG. 7 illustrate examples of manipulating the arm manipulation units viewing an on-screen image that has been rotated 180 degrees according to the related art, and FIG. 8 illustrates an example of manipulating the arm manipulation units while viewing an on-screen image that has been rotated 180 degrees according to an embodiment of the invention.

Referring to FIG. 3, the master robot 1 may include a picture input unit 310, a screen display unit 320, an arm manipulation unit 330, a manipulation signal generator unit 340, a screen-rotation manipulation unit 350, and a control unit 360. The slave robot 2 may include a robot arm 3 and a laparoscope 5.

The picture input unit 310 may receive, over a wired or a wireless network, an image inputted through a camera equipped on the laparoscope 5 of the slave robot 2.

The screen display unit 320 may output an on-screen image, which corresponds to a picture received through the picture input unit 310, as visual information. The screen display unit 320 can be implemented in the form of a monitor 6, etc., and a picture processing process for outputting the received picture through the screen display unit 320 as an on-screen image can be performed by the control unit 360 or by a picture processing unit (not shown).

The arm manipulation unit 330 may enable the operator to manipulate the position and function of the robot arm 3 of the slave robot 2. Although the arm manipulation unit 330 can be formed in the shape of a handle, as exemplified in FIG. 2, the shape is not thus limited and can be implemented in a variety of shapes as long as the same purpose is achieved. Moreover, in another example, one portion can be formed in the shape of a handle, while another portion can be formed in another shape, such as a clutch button, etc.

As described above, the arm manipulation unit 330 can be equipped with a clutch button 14, which can be set to function as a display screen control button. Alternatively, if the laparoscope 5 is not fixed in a particular position to receive a picture but can have its position and/or picture input angle moved or changed according to the operator's adjustment, then the clutch button 14 can also be set to enable the adjusting of the position and/or picture input angle of the laparoscope 5.

When a surgery unit manipulates an arm manipulation unit 330 in order to achieve a position movement or a surgical maneuver by the robot arm 3 and/or the laparoscope 5, the manipulation signal generator unit 340 may generate and transmit a corresponding manipulation signal to the slave robot 2. The manipulation signal can be transmitted and received over a wired or wireless communication, as already described above.

The screen-rotation manipulation unit 350 may serve to receive a command from the operator for rotating the on-screen image in a clockwise or a counterclockwise direction, in cases where the on-screen image outputted through the screen display unit 320 is non-intuitive for the operator.

As exemplified in FIG. 4, the on-screen image on the screen display unit 320 can be displayed rotated by a pre-designated rotation angle (e.g. 15 degrees, 90 degrees, 180 degrees, etc.) in accordance with the number of manipulations on the screen-rotation manipulation unit 350. While the operator would generally input controls such that the on-screen image is rotated in units of 180 degrees (i.e. a rotation such as that illustrated in drawings (a) and (c) of FIG. 4), it will be readily understood from reference to FIG. 4 that the rotation angle by which to rotate the on-screen image is not thus limited.

In this case, the robot arms 3 can have their control conditions automatically renewed, every time the on-screen image is rotated, such that each of the robot arms 3 is controlled by an appropriate arm manipulation unit 330, but since the matching relationship between the robot arms 3 and the arm manipulation units 330 resulting from the rotation of the on-screen image may not be intuitively clear to the operator, it is also conceivable to renew the control conditions according to the designation of the operator (e.g. by clicking a switch button, etc.).

Besides this, it is also possible to have the on-screen image displayed on the screen display unit 320 with the on-screen image being continuously rotated in a pre-designated rotation direction for as long as a manipulation on the screen-rotation manipulation unit 350 is held (e.g. for as long as a pedal 30 is being stepped on), and when the manipulation is stopped, with the on-screen image displayed with a rotation angle tantamount to the rotation up to this time.

If the operator uses the screen-rotation manipulation unit 350 to change the display form of the on-screen image in a desired direction, the positions or functions of the robot arms 3 can be adjusted correspondingly so as to allow the operator conduct surgery in an intuitive manner.

For example, if the operator has made a manipulation to change the display form of the on-screen image, the surgical site and the surgical instrument together would be displayed rotated by a certain rotation angle (e.g. 180 degrees), as exemplified in FIG. 4. Here, it is also possible to provide a position adjustment to always keep the surgical instrument 410 in a certain position (e.g. the position of the surgical instrument illustrated in drawing (a) of FIG. 4), in order that the operator may intuitively recognize which arm manipulation unit 330 controls each surgical instrument 410. This is to control the positions of the robot arms or/and the surgical instruments 410 to match the angle by which the on-screen image displayed on the screen display unit 320 is rotated, so that a surgical instrument 410 positioned on the right side of the display screen can always be manipulated by the operator's right hand.

If the on-screen image is designated to rotate by 180 degrees each time according to the manipulation on the screen-rotation manipulation unit 350, then a surgical instrument 410 that was positioned on the right side in the previous on-screen image and hence manipulated with the right hand may now be positioned on the left side in the current on-screen image, leading to non-intuitive surgery. In this case, it is also possible to configure the settings such that the surgical instrument positioned on the right side of the on-screen image is always manipulated by the operator's right hand, by having the control authority altered between the surgical instruments positioned respectively on the right side and left side with respect to the displayed on-screen image. In this case, compared to the method of moving the surgical instrument to match the rotation angle of the on-screen image, the same results can be expected from merely altering the control authority for each robot arm 3, without having to provide position control for the robot arms 3.

The screen-rotation manipulation unit 350 can be implemented, for example, in the form of a clutch button 14, pedal 30, etc., but its form is not thus limited. For example, the screen-rotation manipulation unit 350 can receive the voice of the operator and analyze the inputted voice to have the on-screen image displayed rotated in a clockwise or counterclockwise direction. Here, a voice recognition technique, etc., can be used for analyzing the voice of the operator.

The control unit 360 may control the actions of each of the component parts so that the functions described above may be implemented. The control unit 360 can also serve to convert a picture inputted through the picture input unit 310 into an on-screen image that will be displayed through the screen display unit 320. Also, the control unit 360 may provide control such that the on-screen image inputted through the screen display unit 320 is displayed rotated in a clockwise or counterclockwise direction when the operator's manipulation is inputted using the screen-rotation manipulation unit 350. Here, the control unit 360 can control the positions of the robot arms 3 in a way that maintains intuitive surgery by the operator or can provide control such that the control authorities for the respective robot arm 3 are exchanged with one another, and can control the manipulation signal generator unit 340 to generate and transmit a corresponding manipulation signal.

A brief description will now be provided, with reference to FIGS. 5 to 8, of a method of manipulating the arm manipulation units 330 according to the rotation manipulation of the on-screen image.

FIG. 5 illustrates an example of an original on-screen image that has not been rotated, as well as the movement directions of each surgical instrument 410a, 410b corresponding to manipulations on the arm manipulation units 330. In this example, it is assumed that the arm manipulation unit 330 includes two handles for individually controlling the movement of the respective surgical instruments 410a, 410b and that the handles are distinguished as handle L (left) and handle R (right) according to their respective positions.

The handle L may control the movement of the surgical instrument 410a positioned on the left side of the on-screen image, while the handle R may control the movement of the surgical instrument 410b positioned on the right side of the on-screen image. Thus, as illustrated by 420a and 420b, pushing the handle L or the handle R forward controls the corresponding surgical instrument to move in an upward direction of the on-screen image.

However, in cases where the original on-screen image is rotated 180 degrees, as in the examples shown in FIG. 6 through FIG. 8, even the arrangement of the surgical instruments 410a and 410b may be displayed rotated 180 degrees from the previous arrangement.

If the movements of the surgical instruments are to be controlled respectively by the handle L or the handle R in this state with the same control conditions as before, then it would be impossible for the operator to conduct surgery in an intuitive manner. This is because the surgical instrument 410b, which was positioned on the right side in the original picture, would be positioned on the left side in the on-screen image that has been rotated 180 degrees, so that the operator would have to predict results that are opposite of the user manipulations. Therefore, if only the on-screen image is displayed rotated 180 degrees as in FIG. 6, then when the operator wishes to move the surgical instrument 410a positioned on the right side in the current on-screen image, the operator would have to pull the handle L, positioned on the left side, towards the operator's body, making it impossible to conduct surgery in an intuitive manner.

The conventional surgical robot system may have a swap function to provide the operator with more convenience in conducting surgery. However, even when the swap function is employed, this merely involves having the surgical instrument 410a, disposed on the right side in the display screen, be controlled by the handle R and having the surgical instrument 410b, disposed on the left side in the display screen, be controlled by the handle R. It does not involve changing the movement or control direction of the surgical instruments 410a, 410b in an intuitive manner. That is, in the example shown in FIG. 7, the surgical instrument 410a disposed on the right side in the display screen may be controlled by the handle R, but when the surgical instrument 410a is to be moved upwards in the display screen, the handle R would have to be pulled towards the operator, so that the operator still has to conduct surgery in a non-intuitive manner.

In contrast, with a surgical robot system according to an embodiment of the invention, not only are the handle L and handle R given the control authorities in a way matching the disposed directions of the surgical instruments in the display screen, as in the example shown in FIG. 8, but also the movement or manipulation directions of the surgical instruments can also be controlled in a manner that is intuitive from the viewpoint of the operator.

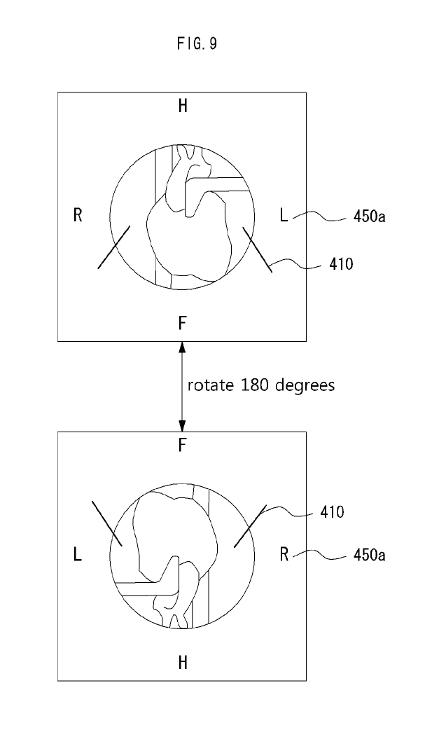

FIG. 9 through FIG. 12 illustrate examples of displaying a direction indicator in correspondence to a rotation of the on-screen image, according to various embodiments of the invention.

As illustrated in each drawing, a direction indicator can be displayed in certain positions among the positions of up, down, left, right, upper left, lower left, upper right, lower right, etc., of the on-screen image displayed on the screen display unit 320.

As in the example shown in FIG. 9, the direction indicator can be composed of one or more letters for indicating a direction, for example, such as H (head), F (foot), L (left), R (right), A (anterior), P (posterior), etc.

Direction indicators composed of letters can be displayed in four positions (e.g. up, down, left, right) or two positions (e.g. right, down), etc., of the display screen (or on-screen image).

In cases where four direction indicators 450a are displayed in their respective positions, as in the example shown in FIG. 9, the direction indicators can include H (head) at the upper side, F (foot) at the lower side, L (left) at the right side, and R (right) at the left side when the original on-screen image is displayed.

However, in an 180 degree-rotated on-screen image, where the original on-screen image is displayed rotated 180 degrees, the direction indicators 450a can include F (foot) at the upper side, H (head) at the lower side, L (left) at the left side, and R (right) at the right side.

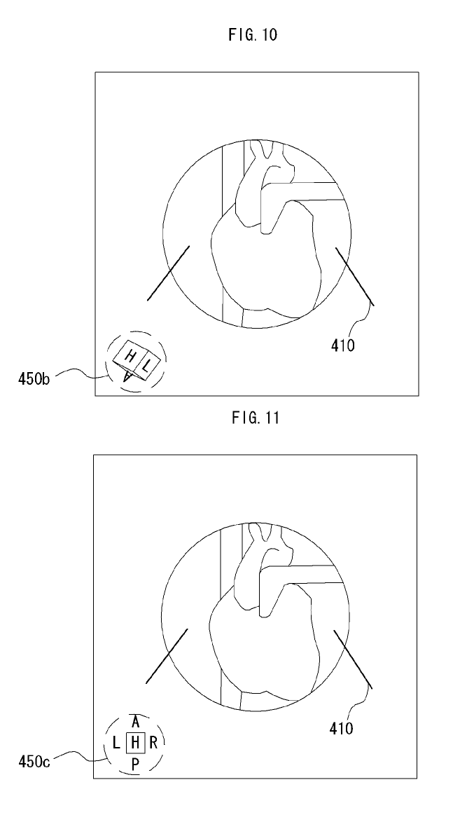

Further, as in the examples shown in FIG. 10 or/and FIG. 11, the rotation direction of the on-screen image can also be shown by the rotation of a hexahedron FIG. 450b that has a direction indicator placed on each side, or by a development drawing 450c of a three-dimensional shape that has a direction indicator placed on each block.

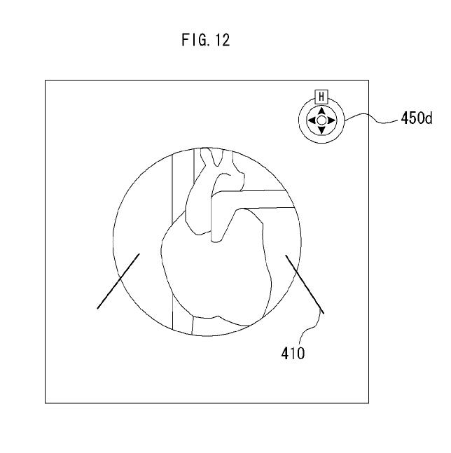

In addition, as in the example shown in FIG. 12, the direction indicator can also be configured in the form of a compass 450d, etc., to display the rotation direction and/or rotation angle in relation to o a base position.

Of course, various methods of displaying a direction indicator can be used, without being limited to the examples above, as long as the operator is able to recognize the direction and rotation angle by which the on-screen image displayed on the screen display unit 320 has been rotated.

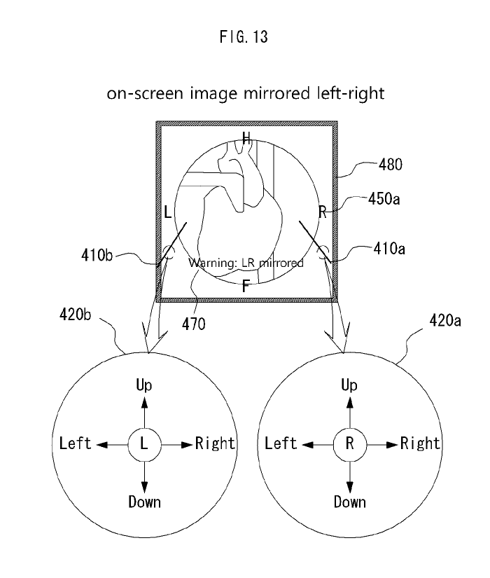

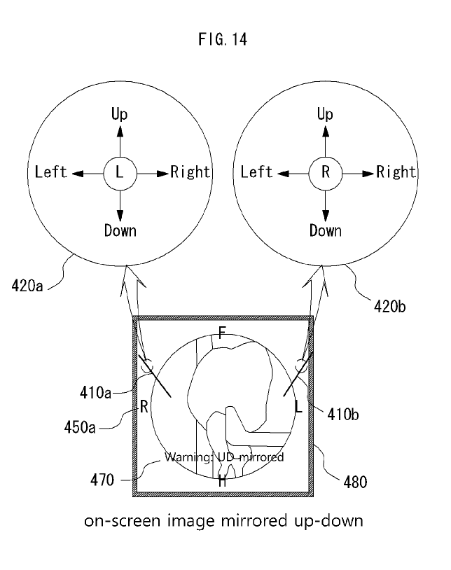

FIG. 13 and FIG. 14 illustrate examples of displaying cautionary information in correspondence to a mirroring of the on-screen image, according to various embodiments of the invention.

While the on-screen image can be displayed rotated in a certain direction on the screen display unit 320 as described above, it can also be displayed mirrored up-down or left-right on the screen display unit 320, as in the examples shown in FIGS. 13 and 14, according to the operator's manipulation or an automatic recognition method as described in this specification.

In this case also, the robot arms 3 may be controlled such that a surgical instrument positioned on the left side in the displayed on-screen image is controlled by the operator's left hand (e.g. an arm manipulation unit positioned on the operator's left side) and a surgical instrument positioned on the right side in the on-screen image is controlled by the operator's right hand (e.g. an arm manipulation unit positioned on the operator's right side).

Unlike a rotation of the picture image, however, an up-down or left-right mirroring can cause confusion for the operator, because the actual geometry of the body is changed. In order to prevent this, there may be a need to notify the operator that a mirrored picture image is shown, by displaying a warning message 470 or cautionary information in the display screen.

In FIG. 13 and FIG. 14, a boundary 480 of a particular color is displayed around the picture, as one example of cautionary information. The cautionary information can also be applied as sound information, etc.

Besides this, direction indicators 450a can be displayed as well, in order that the operator may clearly recognize and be cautioned of the mirroring of the on-screen image.

Since the warning message 470 and/or boundary 480 described above can conceal the surgical site and hinder the conducting of surgery, a method of making the warning message 470 and/or boundary 480 semitransparent can further be employed.

Furthermore, the warning message 470 can be displayed in an area of the screen display unit 320 outside the area in which the picture photographed by the surgical endoscope is displayed, so that the warning message 470 is displayed without overlapping the surgical site. Also, the warning message 470 can be displayed to blink in a certain cycle, so that the operator may clearly recognize the picture mirroring.

This manner of control that enables the operator to control the surgical instrument intuitively according to state of the screen display, as in the detailed description above, will be referred to herein as “display-based instrument control” for convenience.

To provide a brief explanation of display-based instrument control, the movement of a surgical instrument remains consistent with its disposition in the display screen no matter which state the on-screen image of the display screen is in (e.g. in a rotated state, mirrored state, etc.). That is, regardless of the rotating or mirroring of the on-screen image, the operator can consistently control the surgical instrument seen on the right side of the screen with the right hand and control the surgical instrument seen on the left side of the screen with the left hand.

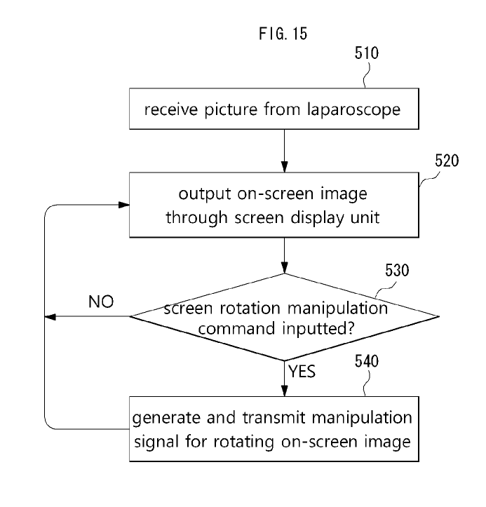

FIG. 15 is a flowchart illustrating a method of controlling a display screen according to an embodiment of the invention.

In describing the method of controlling a display screen according to this embodiment, each step can be performed individually by each component part described with reference to FIG. 3, but for convenient description and understanding, it will be generally assumed that the master robot 1 performs the steps.

Referring to FIG. 15, the master robot 1 may receive a picture from the laparoscope 5 over a wired or wireless network, in step 510, and may output an on-screen image corresponding to the received picture through the screen display unit 320, in step 520.

In step 530, the master robot 1 may determine whether or not a rotation manipulation command using the screen-rotation manipulation unit 350 is inputted from the operator.

If there is no rotation manipulation command inputted, the process returns to step 520, and the operator would conduct surgery for the surgical site by referring to the on-screen image displayed through the screen display unit 320.

However, if there is a rotation manipulation command inputted, the process proceeds to step 540 to provide control such that the on-screen image displayed through the screen display unit 320 is displayed rotated by a certain rotation angle. Here, the master robot 1 can generate a manipulation signal that allows the robot arms 3 to be controlled by the operator in a way matching the rotation of the on-screen image, and can transmit the manipulation signal to the slave robot 2.

A brief description will now be provided on the procedures by which the master robot 1 controls the rotation of the on-screen image and the robot arms 3 according to the manipulation of the screen-rotation manipulation unit 350 by the operator.

First, control can be provided such that the on-screen image displayed through the screen display unit 320 is rotated by a pre-designated rotation angle that is proportional to the number of manipulations on the screen-rotation manipulation unit 350.

Here, the surgical instrument can be controlled to be positioned rotated by a corresponding rotation angle, and the manipulation signal for providing such control can be generated by the manipulation signal generator unit 340 and transmitted to the slave robot 2. Of course, even when the surgical instrument is kept at the current position, as described with reference to FIG. 4, etc., the control authority of each surgical instrument can be transferred to correspond to the arm manipulation unit that can be intuitively recognized by the operator, and the movement and manipulation direction of the robot arm or/and the surgical instrument can be controlled by an intuitive manipulation direction entered by the operator on the arm manipulation unit. In this way, the operator can conduct surgery on a patient using an on-screen image that is rotated to allow intuitive recognition and surgical instruments that are manipulated and controlled by an intuitive method.

If the on-screen image is set to rotate in units of 180 degrees in proportion to the number of manipulations on the screen-rotation manipulation unit 350, then the surgical instruments manipulated by the operator's right hand and left hand, respectively, can be displayed with the left and right reversed, when the on-screen image is displayed rotated 180 degrees. Here, the control unit 360 or the manipulation signal generator unit 340 can make it so that the control authorities of the respective surgical instruments (i.e. the robot arms 3) are exchanged with each other such that the surgical instrument positioned on the right side of the on-screen image (i.e. the surgical instrument that was previously manipulated by the operator's left hand) is manipulated by the operator's right hand, or can generate manipulation signals that provide the same results without actually exchanging control authorities (e.g. manipulation signals by which a manipulation by the operator's right hand controls an arm that is positioned on the right side within the on-screen image but is actually positioned on the left side of the patient) and transmit them to the slave robot 2. This can be applied in the same manner to surgical instruments that are positioned on the left side and right side with respect to a central vertical line in an on-screen image that is displayed rotated, regardless of the rotation angle. Thus, even when the surgical instruments (i.e. the robot arms 3) are actually positioned on the right side and left side of the patient, respectively, the surgical instrument positioned on the right side with respect to the currently displayed on-screen image can always be manipulated by the operator's right hand, making it possible to readily perform surgery based on intuitive recognition by the operator.

Next, the on-screen image displayed through the screen display unit 320 can be rotated in a certain rotating speed in proportion to a manipulation holding time on the screen-rotation manipulation unit 350, and the on-screen image can be controlled to be displayed through the screen display unit 320 after being rotated by the rotation angle at the time the manipulation is stopped. Here, instead of controlling the rotation of the on-screen image according to the manipulation on the screen-rotation manipulation unit 350 by software means, it is also possible to obtain the same effects by having a surgical endoscope, which extends along one direction, be rotated mechanically about an axis formed along its extending direction.

In this case also, the surgical instruments can be made to rotate by a corresponding rotation angle, or the surgical instruments positioned on the left side and right side with respect to a center line of the on-screen image can be controlled respectively by the operator's left hand and right hand in an intuitive manner, as described above.

As described above, the operator remotely controlling the slave robot 2 for surgery on a patient can conduct surgery smoothly by viewing the on-screen image, which is recognized intuitively.

The above descriptions focused on examples of a method in which the operator manipulates the screen-rotation manipulation unit 350 manually or via voice recognition to rotate the on-screen image displayed, and the robot arms 3 are controlled correspondingly.

However, the invention can further include embodiments in which the application of the display conditions for the on-screen image and the control of the robot arms 3 are processed automatically.

That is, an arrangement can be made in which the disposition of a surgical instrument in the on-screen image displayed through the screen display unit 320 can be recognized, based on which the rotation angle of the on-screen image can be automatically computed and applied. Here, image analysis technology can be applied for analyzing the on-screen image inputted through the picture input unit 310 and processed. This technique for rotating the on-screen image will be referred to herein as “instrument-based display control” for convenience.

To provide a brief explanation of instrument-based display control, the position of the surgical instrument is identified and the display form of the on-screen image is changed (e.g. rotated) so as to display the optimum screen. In other words, no matter how the operator manipulates the surgical instrument, the on-screen image is displayed optimized on the display screen.

An example of instrument-based display control can involve forming a virtual trapezoid or quadrilateral with two surgical instruments as the oblique sides and then rotating the on-screen image such that the top side or the bottom side of the trapezoid or quadrilateral becomes parallel to a horizontal plane of the display screen within a margin of error.

Also, position sensors can be mounted on multiple locations of the robot arms 3 (e.g. certain locations on the joint portions at which the robot arms 3 may bend), and the extending directions of the robot arms 3 can be recognized using measurement values obtained by the position sensors, to automatically determine the control conditions of the robot arms 3 (e.g. such that an arm positioned on the right side with respect to the recognized extending direction is controlled by the operator's right hand) and to designate the on-screen display conditions such that the on-screen image is displayed with the front of the extending direction is positioned at an upper side of the screen display unit 320.

Besides the above, the extending directions of the arms can also be recognized by photographing the robot arms from the ceiling of the operating room and analyzing the photographed images. In this case, a paint of a certain color or/and material can be coated over the robot arms or multiple locations of the robot arms, to facilitate the analysis of the photographed images.

The above descriptions focused on examples of a method in which the on-screen image is displayed rotated by software means, while the robot arm, surgical instrument, endoscope camera, etc., maintain their positions, in order to allow the operator to perform surgery intuitively. Furthermore, the same effects can also be obtained by having the robot arm rotate the endoscope camera about an axis formed along its extending direction. Since the spirit of the invention is applied in substantially the same manner for this case also, detailed descriptions on this will be omitted.

The method of controlling a laparoscopic surgical robot system described above can also be implemented as a software program, etc. The code and code segments forming such a program can readily be inferred by computer programmers of the relevant field of art. Also, the program can be stored in a computer-readable medium and can be read and executed by a computer to implement the above method. The computer-readable medium may include magnetic storage media, optical storage media, and carrier wave media.

While the present invention has been described with reference to particular embodiments, it is to be appreciated that various changes and modifications can be made by those skilled in the art without departing from the spirit and scope of the present invention as defined by the scope of claims set forth below.

1.-51. (canceled)

52. A master interface for a surgical robot, the master interface mounted on a master robot for controlling a slave robot, the slave robot comprising two or more robot arms each having a surgical instrument mounted thereon, the master interface comprising:
a screen display unit configured to display an on-screen image corresponding to a picture signal inputted from a surgical endoscope;
two or more arm manipulation units equipped for controlling the two or more robot arms, respectively; and
a control unit configured to provide control such that the on-screen image is rotated or mirrored in a pre-designated direction according to a user manipulation, and configured to provide control such that control conditions for the robot arm are renewed to match the rotated or mirrored on-screen image.

53. The master interface of claim 52, further comprising:
a manipulation signal generator unit configured to generate a manipulation signal according to a user manipulation on the arm manipulation unit and transmit the manipulation signal to the slave robot, and configured to generate a manipulation signal for one or more of a position adjustment for one or more of a robot arm and a surgical instrument and a changing of the arm manipulation units corresponding respectively to the robot arms in order to renew the control conditions.

54. The master interface of claim 52, wherein the control unit computes a rotation angle by instrument-based display control and provides control such that the on-screen image is displayed rotated to match the computed rotation angle.

55. The master interface of claim 54, wherein the instrument-based display control includes foaming a virtual trapezoid or quadrilateral having two surgical instruments as oblique sides and computing the rotation angle such that a top side or a bottom side of the trapezoid or quadrilateral becomes parallel to a horizontal plane of a display screen within a margin of error.

56. A method of controlling a surgical robot system, the method performed in a master robot for controlling a slave robot, the slave robot comprising two or more robot arms each having a surgical instrument mounted thereon, the method comprising:
displaying an on-screen image corresponding to a picture signal inputted from a surgical endoscope;
receiving as input a manipulation command for instructing a rotation or a mirroring of the on-screen image;
providing control such that the on-screen image is rotated or mirrored in a pre-designated direction according to the manipulation command; and
providing control such that a manipulation signal is generated for one or more of a position adjustment for one or more a robot arm and a surgical instrument and a changing of arm manipulation units corresponding respectively to the robot arms, the manipulation signal is transmitted to the slave robot, and a control condition of the robot arm is renewed to match the rotated or mirrored on-screen image.

57. The method of claim 56, wherein the changing of the arm manipulation units is performed according to display-based instrument control.

58. The method of claim 57, wherein the display-based instrument control includes renewing a configuration such that a surgical instrument located on a right side in the rotated on-screen image is manipulated by an arm manipulation unit located on a right side of an operator, and a surgical instrument located on a left side in the rotated on-screen image is manipulated by an arm manipulation unit located on a left side of an operator.

59. The method of claim 58, wherein a manipulation signal is generated for adjusting a position of one or more of a robot arm and a surgical instrument in a certain direction in accordance with a user manipulation for the position adjustment regardless of a rotation of the on-screen image.

60. The method of claim 56, wherein the position adjustment includes moving the surgical instrument in a rotational movement in the on-screen image to match a rotation angle of the on-screen image.

61. The method of claim 56, wherein a rotation angle of the on-screen image is determined in proportion to a number of times the manipulation command is inputted.

62. The method of claim 56, wherein a rotation angle of the on-screen image is determined in proportion to a holding time during which the manipulation command is inputted.

63. The method of claim 56, wherein the receiving of the manipulation command as input comprises:
receiving as input a user voice; and
generating the manipulation command by recognizing and analyzing the inputted user voice.

64. The method of claim 56, wherein the receiving of the manipulation command as input comprises:
receiving as input a sensing value from each of position sensors located on a plurality of locations of the two or more robot arms;
computing a rotation angle for rotating the on-screen image by using the inputted sensing values to recognize an extending direction of the robot arms; and
receiving the computed rotation angle as the manipulation command.

65. The method of claim 64, wherein the rotation angle is computed such that a front of one or more of the robot arm along the extending direction is positioned facing a particular direction on a display screen.

66. The method of claim 56, wherein the receiving of the manipulation command as input comprises:
receiving as input an image taken by a camera equipped on a ceiling;
computing a rotation angle for rotating the on-screen image by using an extending direction of the robot arms as recognized from analyzing the inputted image; and
receiving the computed rotation angle as the manipulation command.

67. The method of claim 66, wherein a paint is coated over any one of an entire area of, an upper area of, and a plurality of locations of the robot arm, the paint having one or more of a pre-designated color and a pre-designated material.

68. The method of claim 66, wherein the surgical endoscope is rotated about an axis formed along its extending direction so that the on-screen image is displayed rotated to match the rotation angle.

69. The method of claim 56, wherein the providing of the control such that the on-screen image is rotated or mirrored includes providing control such that a direction indicator is displayed, the direction indicator providing information related to a rotation of the on-screen image.

70. A method of controlling a surgical robot system, the method performed in a master robot for controlling a slave robot, the slave robot comprising two or more robot arms each having a surgical instrument mounted thereon, the method comprising:
displaying an on-screen image corresponding to a picture signal inputted from a surgical endoscope;
computing a rotation angle for the on-screen image by instrument-based display control such that an optimum screen is displayed by recognizing a position of the surgical instrument in the on-screen image; and
providing control such that the on-screen is displayed rotated by the computed rotation angle.

71. The method of claim 70, wherein the instrument-based display control includes forming a virtual trapezoid or quadrilateral having two surgical instruments as oblique sides and computing the rotation angle such that a top side or a bottom side of the trapezoid or quadrilateral becomes parallel to a horizontal plane of a display screen within a margin of error.

72. The method of claim 70, wherein the surgical endoscope is rotated about an axis formed along its extending direction so that the on-screen image is displayed rotated to match the rotation angle.
